(19) United States
(12) Patent Application Publication
(10) Pub. No.: US 2016/0213764 A1
(43) Pub. Date: Jul. 28, 2016

(54) COMPOSITE TISSUE CANCER VACCINE

(71) Applicant: Batu Biologics, Inc., San Diego, CA (US)

(72) Inventors: Samuel C. Wagner, San Diego, CA (US); Thomas E. Ichim, San Diego, CA (US); Santosh Kesari, San Diego, CA (US); Amit N. Patel, Salt Lake City, UT (US)

(21) Appl. No.: 15/004,864

(22) Filed: Jan. 22, 2016

Related U.S. Application Data
(60) Provisional application No. 62/106,248, filed on Jan. 22, 2015.

(51) Int. Cl. A61K 39/00; C12N 5/0784; A61K 45/06

(52) U.S. Cl. CPC A61K 39/0011; A61K 45/06; C12N 5/0639; A61K 2039/5156; A61K 2039/5154; A61K 2039/5152; C12N 2502/30

(57) ABSTRACT

Disclosed are composite cancer vaccines, in one embodiment generated through 3-dimensional bioprinting or through inoculation of roller cultures. The utilization of decellularized biological matrices such as placental tissue or subintestinal submucosal tissue is disclosed as a substrate for 3-dimensional tissue culture. In one embodiment tumor cells are assembled with monocytes and/or mesenchymal stem cells to represent in vivo existing tumors. The invention teaches means of generating off-the-shelf tumor vaccines containing antigenic properties similar to in vivo growing tumors, which cannot be currently replicated under existing 2-dimensional tumor culture means of generating cell lines

### CROSS-REFERENCE TO RELATED APPLICATION

This application claims the benefit of U.S. Provisional Application No. 62/106,248 filed on Jan. 22, 2015, the contents of which are incorporated herein by reference in its entirety.

### FIELD OF THE INVENTION

The invention pertains to the field of cancer therapy, more particularly to the field of cancer immunotherapy. Specifically, the invention pertains to means of generating cancer vaccines representative of in vivo growing tumors.

### BACKGROUND OF THE INVENTION

The body's immune defense system protects against microbes as well as the body's defense against other chronic diseases, such as those affecting cell proliferation is mediated by early reactions of the innate immune system and by later responses of the adaptive immune system. Innate immunity involves mechanisms that recognize structures which are, for example, characteristic of the microbial pathogens and that are not present on mammalian cells. Examples of such structures include bacterial liposaccharides (LPS), viral double stranded DNA, and unmethylated CpG DNA nucleotides. The effector cells of the innate immune response system comprise neutrophils, macrophages, and natural killer cells (NK cells). In addition to innate immunity, vertebrates, including mammals, have evolved immunological defense systems that are stimulated by exposure to infectious agents and that increase in magnitude and effectiveness with each successive exposure to a particular antigen. Due to its capacity to adapt to a specific infection or antigenic insult, this immune defense mechanism has been described as adaptive immunity. There are two types of adaptive immune responses, called humoral immunity, involving antibodies produced by B lymphocytes, and cell-mediated immunity, mediated by T lymphocytes.

Two types of major T lymphocytes have been described, CD8+ cytotoxic lymphocytes (CTLs) and CD4 helper cells (Th cells). CD8+ T cells are effector cells that, via the T cell receptor (TCR), recognize foreign antigens presented by class I MHC molecules on, for instance, virally or bacterially infected cells. Upon recognition of foreign antigens, CD8+ cells undergo an activation, maturation and proliferation process. This differentiation process results in CTL clones which have the capacity of destroying the target cells displaying foreign antigens. T helper cells on the other hand, are involved in both humoral and cell- mediated forms of effector immune responses. With respect to the humoral, or antibody immune response, antibodies are produced by B lymphocytes through interactions with Th cells. Specifically, extracellular antigens, such as circulating microbes, are taken up by specialized antigen-presenting cells (APCs), processed, and presented in association with class II major histocompatibility complex (MHC) molecules to CD4+ Th cells. These Th cells in turn activate B lymphocytes, resulting in antibody production. The cell-mediated, or cellular, immune response, in contrast, functions to neutralize microbes which inhabit intracellular locations, such as after successful infection of a target cell. Foreign antigens, such as for example, microbial antigens, are synthesized within infected cells and presented on the surfaces of such cells in association with Class I MHC molecules. Presentation of such epitopes leads to the above-described stimulation of CD8+ CTLs, a process which in turn is also stimulated by CD4+ Th cells. Th cells are composed of at least two distinct subpopulations, termed ThI and Th2 cells. The ThI and Th2 subtypes represent polarized populations of Th cells which differentiate from common precursors after exposure to antigen.

Each T helper cell subtype secretes cytokines that promote distinct immunological effects that are opposed to one another and that cross-regulate each other's expansion and function. Th1 cells secrete high amounts of cytokines, such as interferon (IFN) gamma, tumor necrosis factor-alpha (TNF-alpha), interleukin-2 (IL-2), and IL-12, and low amounts of IL-4. Th1 associated cytokines promote CD8+ cytotoxic T lymphocyte (CTL) activity and are most frequently associated with cell-mediated immune responses against intracellular pathogens. In contrast, Th2 cells secrete high amounts of cytokines such as IL-4, IL-13, and IL-10, but low IFN-gamma, and promote antibody responses. Th2 responses are particularly relevant for humoral responses, such as protection from anthrax and for the elimination of helminthic infections.

Whether a resulting immune response is Th1 or Th2-driven largely depends on the pathogen involved and on factors in the cellular environment, such as cytokines. Failure to activate a T helper response, or the correct T helper subset, can result not only in the inability to mount a sufficient response to combat a particular pathogen, but also in the generation of poor immunity against reinfection. Many infectious agents are intracellular pathogens in which cell-mediated responses, as exemplified by Th1 immunity, would be expected to play an important role in protection and/or therapy. Moreover, for many of these infections it has been shown that the induction of inappropriate Th2 responses negatively affects disease outcome. Examples include M. tuberculosis, S. mansoni, and also counterproductive

Th2-like dominated immune responses. Lepromatous leprosy also appears to feature a prevalent, but inappropriate, Th2-like response. HIV infection represents another example. There, it has been suggested that a drop in the ratio of Th1-like cells to other Th cell populations can play a critical role in the progression toward disease symptoms.

As a protective measure against infectious agents, vaccination protocols for protection from some microbes have been developed. Vaccination protocols against infectious pathogens are often hampered by poor vaccine immunogenicity, an inappropriate type of response (antibody versus cell-mediated immunity), a lack of ability to elicit long-term immunological memory, and/or failure to generate immunity against different serotypes of a given pathogen. Current vaccination strategies target the elicitation of antibodies specific for a given serotype and for many common pathogens, for example, viral serotypes or pathogens. Efforts must be made on a recurring basis to monitor which serotypes are prevalent around the world. An example of this is the annual monitoring of emerging influenza A serotypes that are anticipated to be the major infectious strains.

To support vaccination protocols, adjuvants that would support the generation of immune responses against specific infectious diseases further have been developed. For example, aluminum salts have been used as a relatively safe and effective vaccine adjuvants to enhance antibody responses to certain pathogens. One of the disadvantages of such adjuvants is that they are relatively ineffective at stimulating a cell-mediated immune response and produce an immune response that is largely Th2 biased.

It is now widely recognized that the generation of protective immunity depends not only on exposure to antigen, but also the context in which the antigen is encountered. Numerous examples exist in which introduction of a novel antigen into a host in a non-inflammatory context generates immunological tolerance rather than long-term immunity whereas exposure to antigen in the presence of an inflammatory agent (adjuvant) induces immunity. (Mondino et al., Proc. Natl. Acad. Sci., USA 93:2245 (1996); Pulendran et al., J. Exp. Med. 188:2075 (1998); Jenkins et al., Immunity 1:443 (1994); and Kearney et al., Immunity 1:327 (1994)).

A naturally occurring molecule known to regulate adaptive immunity is CD40. CD40 is a member of the TNF receptor superfamily and is essential for a spectrum of cell-mediated immune responses and required for the development of T cell dependent humoral immunity (Aruffo et al., Cell 72:291(1993); Farrington et al., Proc Natl Acad. Sci., USA 91:1099 (1994); Renshaw et al., J Exp Med 180:1889 (1994)). In its natural role, CD40-ligand expressed on CD4+ T cells interacts with CD40 expressed on DCs or B cells, promoting increased activation of the APC and, concomitantly, further activation of the T cell (Liu et al, Semin Immunol 9:235 (1994); Bishop et al., Cytokine Growth Factor Rev 14:297 (2003)). For DCs, CD40 ligation classically leads to a response similar to stimulation through TLRs such as activation marker upregulation and inflammatory cytokine production (Quezada et al., Annu Rev Immunol 22:307 (2004); O'Sullivan Band Thomas, R Crit. Rev Immunol 22:83 (2003)). Its importance in CD8 responses was demonstrated by studies showing that stimulation of APCs through CD40 rescued CD4-dependent CD8+ T cell responses in the absence of CD4 cells (Lefrancois et al., J. Immunol. 164:725 (2000); Bennett et al., Nature 393:478 (1998); Ridge et al., Nature 393:474 (1998); Schoenberger et al., Nature 393:474 (1998). This finding sparked much speculation that CD40 agonists alone could potentially rescue failing CD8+T cell responses in some disease settings.

Other studies, however, have demonstrated that CD40 stimulation alone insufficiently promotes long-term immunity. In some model systems, anti-CD40 treatment alone insufficiently promoted long-term immunity. In some model systems, anti-CD40 treatment alone can result in ineffective inflammatory cytokine production, the deletion of antigen-specific T cells (Mauri et al., Nat Med 6:673 (2001); Kedl et al., Proc Natl Acad. Sci., USA 98:10811(2001)) and termination of B cell responses (Erickson et al., J Clin Invest 109:613 (2002)). Also, soluble trimerized CD40 ligand has been used in the clinic as an agonist for the CD40 pathway and what little has been reported is consistent with the conclusion that stimulation of CD40 alone fails to reconstitute all necessary signals for long term CD8+ T cell immunity (Vonderheide et al., J Clin Oneal 19:3280 (2001)).

Various agonistic antibodies have been reported by different groups. For example, one mAb CD40.4 (5c3) (PharMingen, San Diego Calif.) has been reported to increase the activation between CD40 and CD40L by approximately 30-40%. (Schlossman et al., Leukocyte Typing, 1995, 1:547-556). Also, Seattle Genetics in U.S. Pat. No. 6,843,989 allege to provide methods of treating cancer in humans using an agonistic anti-human CD40 antibody. Their antibody is purported to deliver a stimulatory signal, which enhances the interaction of CD40 and CD40L by at least 45% and enhances CD4OL-mediated stimulation and possess in vivo neoplastic activity. They obtain this antibody from S2C6, an agonistic anti-human CD40 antibody previously shown to deliver strong growth-promoting signals to B lymphocytes. (Paulie et al., 1989, J. Immunol. 142:590-595).

Because of the activity of CD40 in innate and adaptive immune responses, various CD40 agonists have been explored for usage as vaccine adjuvants and in therapies wherein enhanced cellular immunity is desired. Recently, it was demonstrated that immunization with antigen in combination with some TLR agonists and anti-CD40 treatment (combined TLR/CD40 agonist immunization) induces potent CD8+ T cell expansion, elicting a response 10-20 fold higher than immunization with either agonist alone (Ahonen et al., J Exp Med 199:775 (2004)). This was the first demonstration that potent CD8+ T cell responses can be generated in the absence of infection with a viral or microbial agent. Antigen specific CD8+ T cells elicited by combined TLR/CD40 agonist immunization demonstrate lytic function, gamma interferon production, and enhanced secondary responses to antigenic challenge. Synergistic activity with anti-CD40 in the induction of CD8+ T cell expansion has been shown with agonists of TLRI/6, 2/6, 3, 4, 5, 7 and 9. This suggests that combined TLR/CD40 agonist immunization can reconstitute all of the signals required to elicit profound acquired cell-mediated immunity.

It is known that NKT cells are immunoregulatory T-lymphocytes that express aT cell receptor which is restricted by the non-polymorphic COld antigen presenting molecule. NKT cells recognize the COld- presented glycolipid, alpha-galactosylceramide (a-Gai-Cer). Upon recognition of a-Gai-Cer, NKT cells become activated and produce cytokines including IL-4, IL-10, IL-13 and IFN-'y, and as such, they can either upregulate or downregulate immune responses by promoting the secretion of immune regulatory cytokines. Mice which are devoid of NKT cells are more susceptible to bacterial infections and resistance to some tumors, demonstrating an important role for NKT cells in host defense. It has also been shown that activation of NKT cells by the administration of a-Gai-Cer, as a monotherapy, to mice can enhance the immunity to tumors with limited success (Matsuyoshi, H., S. Hirata, Y. Yoshitake, Y. Motomura, D. Fukuma, A. Kurisaki, T. Nakatsura, Y. Nishimura, and S. Senju. 2005. Therapeutic effect of alpha-galactosylceramide-loaded dendritic cells genetically engineered to express SLC/CCL21along with tumor antigen against peritoneally disseminated tumor cells. Cancer Sci. 96:889-896; Crowe, N.Y., J. M. Coquet, S. P. Berzins, K. Kyparissoudis, R. Keating, D. G. Pellicci, Y. Hayakawa, D. I. Godfrey, and M. J. Smyth. 2005. “Differential antitumor immunity mediated by NKT cell subsets in vivo”, J. Exp. Med. 202:1279-1288; Smyth, M. J., N.Y. Crowe, Y. Hayakawa, K. Takeda, H. Yagita, and D. I. Godfrey. 2002. “NKT cells—conductors of tumor immunity?”, Curr. Opin. Immunol. 14:165-171).

To increase the effectiveness of an adaptive immune response, such as in a vaccination protocol or during a microbial infection, it is therefore important to develop novel, more effective, vaccine adjuvants. The present invention satisfies this need and provides other advantages as well. Previously, the present inventors have reported novel synergistic adjuvants comprising the combination of a toll like receptor (TLR) agonist and a CD40 agonist. These agonists in combination elicit a synergistic effect on cellular immunity. These synergistic adjuvants and the prophylactic and therapeutic applications thereof are disclosed in U.S. Application Publication No. US 20040141950 published on Jul. 22, 2004, and which patent application is incorporated by reference in its entirety herein. This invention relates to the discovery of another synergistic adjuvant combination and the use thereof as a therapeutic or prophylactic immune potentiating combination.

### DETAILED DESCRIPTION OF THE INVENTION

The invention teaches the generation of composite cancer vaccines, in one embodiment generated through 3D bioprinting. In one embodiment tumor cells are assembled with monocytes and/or mesenchymal stem cells to represent in vivo existing tumors. The invention teaches means of generating off-the-shelf tumor vaccines containing antigenic properties similar to in vivo growing tumors, which cannot be currently replicated under existing 2 dimensional tumor culture means of generating cell lines.

In certain other embodiments, said cells are primary culture cells. In another specific embodiment, cells are cells that have been cultured in vitro. In certain other specific embodiments, said cells have been genetically engineered to produce a protein or polypeptide not naturally produced by the cells, or have been genetically engineered to produce a protein or polypeptide in an amount greater than that naturally produced by the cells. Cytokines genetically produced by the cells are cytokines that stimulate immunogenicity or provide cell growth to resemble conditions found in a tumor. In specific embodiments, said protein or polypeptide is a cytokine or a peptide comprising an active part thereof.

In more specific embodiments, said cytokine is one or more of adrenomedullin (AM), angiopoietin (Ang), bone morphogenetic protein (BMP), brain-derived neurotrophic factor (BDNF), epidermal growth factor (EGF), erythropoietin (Epa), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (PIGF), platelet-derived growth factor (PDGF), thrombopoietin (Tpo), transforming growth factor alpha (TGF-a), TGF-, tumor necrosis factor alpha (TNF-a), vascular endothelial growth factor (VEGF), or a Wnt protein. In other specific embodiments, said protein or polypeptide is an interleukin or an active portion thereof. In various more specific embodiments, said interleukin is interleukin-1alpha (IL-Ia), IL-I, IL- IFI,IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, both IL-12 alpha and beta subunits, IL-13, IL-14, IL-15, IL-16, IL-17A, IL-17B, IL-17C, IL-170, IL-17E, IL-17F isoform 1, IL-17F isoform 2, IL-18, IL-19, IL-20, IL-21, IL-22, IL-23 p19 subunit, IL-23 p40 subunit, IL-23 p19 subunit and IL-23 p40 subunit together, IL-24, IL-25, IL-26, IL-27B, IL-27-p28, IL-27B and IL-27-p28 together, IL-28A, IL-28B, IL-29, IL-30, IL-31, IL-32, IL-33, IL-34, IL-35, IL-36α, IL-36 β, IL-36γ.

“Composite cell”, “composite cellular vaccine”, “3d cellular vaccine,” as used herein, means a combination of at least one type of cell and placental vascular scaffold or portion thereof, wherein the combination resembles an in vivo tumor. In certain embodiments, the placental vascular scaffold of the composite described herein comprises decellularized human placental vascular scaffold (DHPVS). In certain embodiments, the composite cellular vaccine described herein comprise an entire DHPVS, that is, an entire human placenta comprising placental vasculature that has been decellularized in accordance with the methods described herein and seeded with tumor cell lines. In certain embodiments, the composite cellular vaccine described herein comprise a portion of a placenta, e.g., a portion of a DHPVS. In a specific embodiment, the composite cellular vaccine described herein comprise a portion of a placenta, e.g., a portion of a DHPVS, wherein said portion comprises one or more regions of the placenta that comprise vasculature, e.g., one or more cotyledons, which are separations of the decidua basalis of the placenta that comprise distinct vascular domains. In another specific embodiment, the composite cellular vaccine described herein comprise a portion of a placenta, e.g., a portion of a DHPVS, wherein said portion comprises a portion of the placenta that has been removed from the remainder of the placenta and decellularized according to the methods described herein, either prior or subsequent to such removal from the remainder of the placenta. For example, the portion is of a desired size and shape, e.g., a cube, that has been removed from (e.g., excised out of or stamped out of) the placenta (e.g., the DHPVS).

In certain embodiments, the methods of generating composite cellular vaccine described herein comprise bioprinting of one or more cell types onto or into decellularized placental vascular scaffold.

“Bioprinting,” as used herein, generally refers to the deposition of material, such as living cells, and, optionally, other components (e.g., extracellular matrix; synthetic matrices) onto a surface using standard or modified printing technology, e.g., ink jet printing technology. Basic methods of depositing cells onto surfaces, and of bioprinting cells, including cells in combination with hydrogels, are described in Warren et al. U.S. Pat. No. 6,986,739, Boland et al. U.S. Pat. No. 7,051,654, Yoo et al., U.S. Patent Application Publication No. 2009/0208466 and Xu et al., U.S. Patent Application No. 2009/0208577, the disclosures of each of which are incorporated by reference herein their entirety. Additionally, bioprinters useful for production of the composite cellular vaccine provided herein are commercially available, e.g., the 30-Bioplotter™ from Envisiontec GmbH (Giadbeck, Germany); and the NovoGen MMX Bioprinter™ from Organovo (San Diego, Calif.).

Provided herein is a composite cellular cancer vaccine comprising one or more types of cells, and decellularized placental vascular scaffold, or other types of scaffolds including extracellular matrix from tissues suitable for cellular seeding.

Decellularized placental vascular scaffold comprises substantially intact placental vasculature matrix; that is, the structure of the vasculature of the placenta from which the matrix is obtained is substantially preserved during decellularization and subsequent production of the cellular composite cancer vaccine. In certain embodiments, once the cellular composite vaccine is generated, the cells are irradiated and implanted with adjuvant or without adjuvant to stimulate immunity to cancer.

In one embodiment the practice of the invention involves obtaining a human placenta that is recovered shortly after its expulsion after normal birth, or after a Caesarian section. The placenta is recovered from a patient after informed consent and after a complete medical history of the patient is taken and is associated with the placenta. Preferably, the medical history continues after delivery. Such a medical history can be used to coordinate subsequent use of the placenta or the stem cells harvested therefrom. For example, human placental stem cells can be used, in light of the medical history, for personalized medicine for the infant associated with the placenta, or for parents, siblings or other relatives of the infant. The umbilical cord blood and placental blood are removed, and can be used for other purposes or discarded. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., by LifeBank USA, Cedar Knolls, N.J. Preferably, the placenta is gravity drained without further manipulation so as to minimize tissue disruption during cord blood recovery. Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between about 20° C. to about 28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. Pat. No. 7,147,626. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta. The placenta can be stored under sterile conditions and at either room temperature or at a temperature of 5° C. to 25° C. The placenta may be stored for a period of for a period of four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta is preferably stored in an anticoagulant solution at a temperature of 5° C. to 25° C. Suitable anticoagulant solutions are well known in the art, e.g., a solution of heparin or warfarin sodium. In a preferred embodiment, the anticoagulant solution comprises a solution of heparin (e.g., 1% w/w in 1:1000 solution). The exsanguinated placenta is preferably stored for no more than 36 hours before placental stem cells are collected.

In certain embodiments, the composite cellular vaccine described herein comprises only a portion of a decellularized placenta obtained in accordance with the above-described methods and seeded with cancer cell lines. Said cell lines include K-S62, THP-1J82, RT4, ScaBER, T24, TCCSUP, S637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-1S7 Breast medulla, carcinoma, pleural effusion, MDA-MB-17S- VII Breast, ductal carcinoma, pleural Effusion, MDA-MB-361Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph node ME-180 Epidermoid carcinoma, cervix, metastasis to omentum, MEL-17S Melanoma, MEL-290 Melanoma, HLA-A*0201Melanoma cells, MS7S1Epidermoid carcinoma, cervix, metastasis to lymph Node, SiHa Squamous carcinoma, cervix, JEG-3 Choriocarcinoma, Caco-2 Adenocarcinoma, colon HT-29 Adenocarcinoma, colon, moderately well-differentiated grade II, SK-C0-1Adenocarcinoma, colon, ascites, HuTu 80 Adenocarcinoma, duodenum, A-2S3 Epidermoid carcinoma, submaxillary gland FaDu Squamous cell carcinoma, pharynx, A-498 Carcinoma, kidney, A-704 Adenocarcinoma, kidney Adenocarcinoma, kidney, SK-HEP-1Adenocarcinoma, liver, ascites, A-427 Carcinoma, lung, Caki-1Clear cell carcinoma, consistent with renal primary, metastasis to skin, Caki-2 Clear cell carcinoma, consistent with renal primary, SK-NEP-1Wilms' tumor, pleural effusion, SW 839 Adenocarcinoma, kidney, SK-HEP-1Adenocarcinoma, liver, ascites, A-427 Carcinoma, lung Calu-1Epidermoid carcinoma grade III, lung, metastasis to pleura, Calu-3 Adenocarcinoma, lung, pleural effusion, Calu-6 Anaplastic carcinoma, probably lung, SK-LU-1Adenocarcinoma, lung consistent with poorly differentiated, grade III, SK-MES-1Squamous carcinoma, lung, pleural effusion, SW 900 Squamous cell carcinoma, lung, EB1 Burkitt lymphoma, upper maxilia, EB2 Burkitt lymphoma, ovary P3HR-1Burkitt lymphoma, ascites, HT-144 Malignant melanoma, metastasis to subcutaneous tissue Malme-3M Malignant melanoma, metastasis to lung, RPMI-79S1Malignant melanoma, metastasis to lymph node, SK-MEL-1Malignant melanoma, metastasis to lymphatic system, SK-MEL-2 Malignant melanoma, metastasis to skin of thigh, SK-MEL-3 Malignant melanoma, metastasis to lymph node SK-MEL-S Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1Adenocarcinoma, pancreas, metastasis to liver, Capan-2 Adenocarcinoma, pancreas, DU 14S Carcinoma, prostate, metastasis to brain, A-204 Rhabdomyosarcoma, Saos-2 Osteogenic sarcoma, primary, SK-ES-1 Anaplastic osteosarcoma versus Swing sarcoma, SK-LNS-1Leiomyosarcoma, vulva, primary, SW 684 Fibrosarcoma, SW 872 Liposarcoma SW 982 Axilla synovial sarcoma, SW 13S3 Chondrosarcoma, humerus, U-2 OS Osteogenic sarcoma, bone primary, Malme-3 Skin fibroblast, KATO III Gastric carcinoma, Cate-1B Embryonal carcinoma, testis, metastasis to lymph node, Tera-1Embryonal carcinoma, Tera-2 Embryonal carcinoma, SW579 Thyroid carcinoma, AN3 CA Endometrial adenocarcinoma, metastatic, HEC-I-A Endometrial adenocarcinoma HEC-1-B Endometrial adenocarcinoma, SK-UT-1 Uterine, mixed mesodermal tumor, consistent with leiomyosarcomagrade III, SK-UT-IB Uterine, mixed mesodermal tumor, Sk-Me128 Melanoma SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast BT-549 Ductal carcinoma, breast, DU4475 Metastatic cutaneous nodule, breast carcinoma HBL-100 Breast, Hs 578Bst Breast, Hs 578T Ductal carcinoma, breast, MDA-MB-330 Carcinoma, breast MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738BIBladder, normal fetus NIH:OVCAR-3 Ovary, adenocarcinoma, Hs 67 Thymus, normal, RD-ES Ewing's sarcoma ChaGo K-1Bronchogenic carcinoma, subcutaneous, metastasis, human, WERI-Rb-1 Retinoblastoma NCI-H446 Small cell carcinoma, lung, NCI-H209 Small cell carcinoma, lung, NCI-H146 Small cell carcinoma, lung, NCI-H441Papillary adenocarcinoma, lung, NCI-H345 Small cell carcinoma, lung, NCI- H820 Papillary adenocarcinoma, lung, NCI-H520 Squamous cell carcinoma, lung, NCI-H661Large cell carcinoma, lung NCI-H510A Small cell carcinoma, extra-pulmonary origin, metastatic D283 Med Medulloblastoma Daoy Medulloblastoma, D341Med Medulloblastoma, AML-193 Acute monocyte leukemia MV4-11 Leukemia biphenotype, NCI-H82 Small cell carcinoma, lung H9 T-celllymphoma, NCI-H460 Large cell carcinoma, lung, NCI-H596 Adenosquamous carcinoma, lung NCI-H676B Adenocarcinoma of lung. Furthermore, the placenta may be manipulated to obtain the desired portion, e.g., to obtain a desired placental circulatory unit (e.g., a cotyledon) before the portion of the placenta is further processed (e.g., processed as described herein, e.g., decellularized).

In certain embodiments, when only a portion of a placenta is used in the generation of the organoids described herein, the entire placenta is processed as desired (e.g., decellularized as described below), followed by isolation of the specific portion of the placenta to be used (e.g., by cutting or stamping out the desired portion of the placenta from the whole processed placenta). Once the placenta is prepared as above, and optionally perfused, it is decellularized in such a manner as to preserve the native structure of the placental vasculature, e.g., leave the placental vasculature substantially intact. As used herein, “substantially intact” means that the placental vasculature remaining after decellularization retains all, or most, of the gross structure of the placental vasculature prior to decellularization. In certain embodiments, the placental vasculature is capable of being re-seeded, e.g., with vascular endothelial cells or other cells, specifically tumor cell lines so as to recreate the tumor vasculature. The Placental tissue may be sterilized, e.g., by incubation in a sterile buffered nutrient solution containing antimicrobial agents, for example an antibacterial, an antifungal, and/or a sterilant compatible with the transplant tissue. The sterilized placental tissue may then be cryopreserved for further processing at a later time or may immediately be further processed according to the next steps of this process including a later cryopreservation of the tissue matrix or other tissue products of the process.

Means of decellularizing tissue including physical, chemical, and biochemical methods. See, e.g. U.S. Pat. No. 5,192,312 (Orton) which is incorporated herein by reference. Such methods may be employed in accordance with the process described herein. However, the decellularization technique employed preferably does not result in gross disruption of the anatomy of the placental tissue or substantially alter the biomechanical properties of its structural elements, and preferably leaves the placental vasculature substantially intact. In certain embodiments, the treatment of the placental tissue to produce a decellularized tissue matrix does not leave a cytotoxic environment that mitigates against subsequent repopulation of the matrix with cells that are allogeneic or autologous to the recipient. As used herein, cells and tissues that are “allogeneic” to the recipient are those that originate with or are derived from a donor of the same species as a recipient of the placental vascular scaffold, and “autologous” cells or tissues are those that originate with or are derived from a recipient of the placental vascular scaffold.

In one embodiment the placental tissue is cryopreserved in the presence of one or more cryoprotectants. Colloid-forming materials may be added during freeze-thaw cycles to alter ice formation patterns in the tissue. For example, polyvinylpyrrolidone (10% w/v) and dialyzed hydroxyethyl starch (10% w/v) may be added to standard cryopreservation solutions (DMEM, 10% DMSO, 10% fetal bovine serum) to reduce extracellular ice formation while permitting formation of intracellular ice. In some embodiments, the placental tissue is decellularized using detergents or combinations thereof, for example, a nonionic detergent, e.g., Triton X-100, and an anionic detergent, e.g., sodium dodecyl sulfate, may disrupt cell membranes and aid in the removal of cellular debris from tissue. Preferably, residual detergent in the decellularized tissue matrix is removed, e.g., by washing with a buffer solution, so as to avoid interference with the later repopulating of the tissue matrix with viable cells.

In one embodiment the means of decellularization is performed by the administration of a solution effective to lyse native placental cells. Preferably, the solution is an aqueous hypotonic or low ionic strength solution formulated to effectively lyse the cells. In certain embodiments, the aqueous hypotonic solution is, e.g. deionized water or an aqueous hypotonic buffer. In specific embodiments, the aqueous hypotonic buffer contains one or more additives that provide sub-optimal conditions for the activity of one or more proteases, for example collagenase, which may be released as a result of cellular lysis. Additives such as metal ion chelators, for example 1,10-phenanthroline and ethylenediaminetetraacetic acid (EDTA), create an environment unfavorable to many proteolytic enzymes. In other embodiments, the hypotonic lysis solution is formulated to eliminate or limit the amount of divalent cations, e.g., calcium and/or zinc ions, available in solution, which would, in turn, reduce the activity of proteases dependent on such ions. Perfusion of the placental vasculature may be performed according to the methods described in U.S. Pat. No. 8,057,788.

It is important to prevent formation of viscous liquids during the decellularlization process, accordingly, in some embodiments, decellularization of placental tissue includes treatment of the tissue with one or more nucleases, e.g., effective to inhibit cellular metabolism, protein production and cell division without degrading the underlying collagen matrix. Nucleases that can be used for digestion of native cell DNA and RNA include either or both of exonucleases or endonucleases. Suitable nucleases for decellularization are commercially available. For example, it is known that exonucleases that effectively inhibit cellular activity include DNAase I (SIGMA Chemical Company, St. Louis, Mo.) and RNAase A (SIGMA Chemical Company, St. Louis, Mo.) and endonucleases that effectively inhibit cellular activity include EcoRI (SIGMA Chemical Company, St. Louis, Mo.) and Hind III (SIGMA Chemical Company, St. Louis, Mo.). For the practice of the invention, selected nucleases may be contained in a physiological buffer solution which contains ions that are optimal for the activity of the nuclease, e.g., magnesium salts or calcium salts. It is also preferred that the ionic concentration of the buffered solution, the treatment temperature and the length of treatment are selected to assure the desired level of effective nuclease activity. The buffer is preferably hypotonic to promote access of the nucleases to cell interiors.

In certain embodiments, the one or more nucleases comprise DNAase I and RNAase A. Preferably, the nuclease degradation solution contains about 0.1 microgram/mL to about 50 microgram/mL, or about 10 microgram/mL, of the nuclease DNAase I, and about 0.1 microgram/mL to about 10 microgram/mL preferably about 1.0 microgram/mL, of RNAase A. The placental tissue may be decellularized by application of the foregoing enzymes at a temperature of about 20° C. to 38° C., preferably at about 3rc., e.g., for about 30 minutes to 6 hours.

It is known in the art that the process of decellularization is associated with creation of tissue debris, therefore the placental tissue matrix in certain embodiments is washed in a wash solution to assure removal of cell debris which may include cellular protein, cellular lipids, and cellular nucleic acid, as well as any extracellular debris. Removal of this cellular and extracellular debris reduces the likelihood of the transplant tissue matrix eliciting an adverse immune response from the recipient upon implant. For example, the tissue may be washed one or more times with a wash solution, wherein the wash solution is, e.g., PBS or Hanks' Balanced Salt Solution (HBSS). The composition of the balanced salt solution wash, and the conditions under which it is applied to the transplant tissue matrix may be selected to diminish or eliminate the activity of proteases or nucleases utilized during the decellularization process. In specific embodiments, the wash solution does not contain magnesium or calcium, e.g. magnesium salts or calcium salts, and the washing process proceeds at a temperature of between about 2° C. and 42° C., e.g., 4° C. most preferable. The transplant tissue matrix may be washed, e.g., incubated in the balanced salt wash solution for up to 1, 2, 3, 4, 5, 6, 7, 8, 9, 10, 11 or 12 days, e.g., with changes in wash solution every −13 days. Optionally, an antibacterial, an antifungal or a sterilant or a combination thereof, may be included in the wash solution to protect the transplant tissue matrix from contamination with environmental pathogens. Washing may be performed by soaking the placental tissue with or without mild agitation.

To allow for large scale production, it may not be feasible to seed the tissue the same day that the cells are added. Accordingly, the placental tissue matrix, once decellularized, can be preserved by cryopreservation. Techniques of cryopreservation of tissue are well known in the art. See, e.g., Brockbank, K. G. M., “Basic Principles of Viable Tissue Preservation,” In: Transplantation Techniques and Use of Cryopreserved Allograft Cardiac Valves and Vascular Tissue, D. R. Clarke (ed.), Adams Publishing Group, Ltd., Boston. pp 9-23 (discussing cryopreservation of tissues and organs). The tissue matrix, whether or not having been cryopreserved, in certain embodiments is treated to enhance the adhesion and inward migration of the allogeneic or autologous cells, in vitro, which will be used to repopulate the transplant tissue.

In certain embodiments, attachment of autologous or allogeneic cells to decellularized placental vascular scaffold may be increased, e.g., by contacting the placental vascular scaffold with serum (human or fetal bovine, maximal binding with 1% serum) and/or purified fibronectin, e.g., in culture medium in which the decellularized placental vascular scaffold is placed, e.g., in preparation for repopulation with allogeneic or autologous cells. Each of the two homologous subunits of fibronectin has two cell recognition regions, including one comprising the Arg-Giy-Asp (RGD) sequence. A second site, binding glycosaminoglycans, acts synergistically and appears to stabilize the fibronectin-cell interactions mediated by the RGD sequence. Additionally, platelet rich plasma, or platelet lysate may be utilized. As such, in a specific embodiment, the decellularized placental vascular scaffold is contacted with both fibronectin and a glycosaminoglycan, e.g., heparin, for a period effective for binding of the fibronectin to surfaces of the placental vascular scaffold to be repopulated with allogeneic or autologous cells. The fibronectin, and optionally glycosaminoglycan, can be included within a physiologically—acceptable buffer or culture medium, e.g., sodium phosphate/glycerin/bovine serum albumin and Dulbecco's Modified Eagle's Medium (DMEM) (e.g., GIBCO). The buffer or culture medium is preferably maintained at a physiologically acceptable pH, e.g., about 6.8 to 7.6. Fibronectin may be obtained from human blood, processed to limit contamination with virus, or may be obtained from commercial sources. The concentration of fibronectin and/or glycoprotein may range from about 1 microgram/mlto about 100 microgram/ml,e.g., about 10 microgram/ml. The preferred weight ratio of fibronectin to heparin is about 100:1 to about 1:100, or about 10:1to about 1:10, e.g., 10:1 ibronectin:glycosaminoglycan, e.g. heparin. The decellularized placental vascular scaffold may be contacted with, e.g., treated with, one or more compositions that act, e.g., to enhance cell chemotaxis, increasing the rate of directional movement along a concentration gradient of the substance in solution. With respect to fibroblast cells, fibroblast growth factor, platelet-derived growth factor, transforming growth factor-beta (TGF-.beta.), fibrillar collagens, collagen fragments, and fibronectin are chemotactic.

In a specific, preferred embodiment, the placenta is decellularized as follows. Placental tissue, e.g., a whole placenta or lobule (cotyledon) of a placenta, from which blood has been removed is first frozen at −20° C. to −180° C., e.g., about-80° C., e.g., for about 24 hours. The tissue is then thawed at about 4° C. overnight. The thawed tissue is then digested with 0.1% trypsin at room temperature for 2 hours to 24 hours to produce digested placental tissue at 25° C. to about 37° C. In this digestion, and in subsequent steps, solution is passed through the placental vasculature (perfusion decellularization). The digested tissue is then treated sequentially with 1%, 2% and 3% Triton-X100 for 24 hours each at room temperature or about 25° C. The Triton-X100 treatments are then followed by treatment of the tissue with 0.1% SDS-PBS for 24 hat room temperature or at about 25° C., after which the cellular material is substantially removed. The tissue is then extensively washed with 1-10 changes of phosphate buffered saline (PBS), followed by treatment with DNase I (150 U/mL) for 1hour at room temperature, each step at room temperature or about 25° C. Finally, the remaining decellularized placental vascular scaffold is again extensively washed at room temperature or about 25° C. with PBS+1% antibiotics (penicillin+streptomycin), optionally dried, and preserved at 4° C.

In order to alter biological properties, including immunogenicity, it may be important to add to the decellularized matrix various biocompatible or immunogenic scaffold or stabilizing materials. In the practice of the invention, following decellularization, the resulting placental vascular scaffold may be combined with one or more synthetic matrices, e.g., synthetic polymers. In a specific embodiment, the synthetic matrix stabilizes the three-dimensional structure of the placental vascular scaffold, e.g., to facilitate production of the composite cellular vaccine. In another specific embodiment, said synthetic matrix comprises a polymer or a thermoplastic. In a more specific embodiment, said synthetic matrix is a polymer or a thermoplastic. In more specific embodiments, said thermoplastic is polycaprolactone, polylactic acid, polybutylene terephthalate, polyethylene terephthalate, polyethylene, polyester, polyvinyl acetate, or polyvinyl chloride. In other more specific embodiments, said polymer is polyvinylidine chloride, poly(o-carboxyphenoxy)-p-xylene) (poly(o-CPX)), poly(lactide-anhydride) (PLAA), n-isopropyl acrylamide, acrylamide, pent erythritol diacrylate, polymethyl acrylate, carboxymethylcellulose, or poly(lactic-co-glycolic acid) (PLGA). In another more specific embodiment, said polymer is polyacrylamide. In another embodiment subintestinal mucosa may be used. Alternatively various molecular weight form of hyaluronic acid may be utilized.

Tumor cell lines or healthy tumor-associated cells may be loaded onto the decellularized placental vascular scaffold by any physiologically-acceptable method. In certain embodiments, the cells are suspended in, e.g., a liquid culture medium, salt solution or buffer solution, and the cell-containing liquid is perfused into the placental vascular scaffold through one or more of the vascular matrices. The placental vascular scaffold may also be cultured in such a cell-containing liquid culture medium, salt solution or buffer solution for a time sufficient for a plurality of the cells to attach to said placental vascular scaffold. Cells may also be loaded onto the placental vascular matrix by seeding on the surface of the scaffold, or by injecting cells into the vessels using, e.g., a needle or an infusion pump. In certain embodiments, cells are loaded onto the decellularized placental vascular scaffold by bioprinting.

In certain embodiments after cells are loaded onto a decellularized placental vascular scaffold, the cells and scaffold are cultured for a desired period of time. In a specific embodiment, the cells and scaffold are cultured in a roller bioreactor.

To provide angiogenic support for the tumor cells, as well as to facilitate growth factor production, monocytes may be added, monocytes may be first precultured to induce a M2 phenotype. In certain other embodiments isolated stem cells or progenitor cells. In specific embodiments, said isolated stem cells or progenitor cells are isolated embryonic stem cells, embryonic germ cells, induced pluripotent stem cells, mesenchymal stem cells, bone marrow-derived mesenchymal stem cells, bone marrow- derived mesenchymal stromal cells, tissue plastic-adherent placental stem cells (PDAC.®.), umbilical cord stem cells, amniotic fluid stem cells, amnion derived adherent cells (AMDACs), osteogenic placental adherent cells (OPACs), adipose stem cells, limbal stem cells, dental pulp stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells, or side population stem cells. In other specific embodiments, the one or more types of cells comprised within the organoids are, or comprise, isolated hematopoietic stem cells or hematopoietic progenitor cells. In other specific embodiments, the one or more types of cells comprised within the organoids are tissue culture plastic-adherent CD34.sup.−, CD10.sup.+, CD105.sup.+, and CD200.sup.+placental stem cells, e.g., the placental stem cells described in U.S. Pat. No. 7,468,276 and U.S. Pat. No. 8,057,788, the disclosures of which are hereby incorporated by reference in their entireties. In a specific embodiment, said placental stem cells are additionally one or more of CD45.sup.−, CD80.sup.−, CD86.sup.−, or CD90.sup.+. In a more specific embodiment, said placental stem cells are additionally CD45.sup.−, CD80.sup.−, CD86.sup.−, and CD90 positive.

In one specific embodiment, placentas are pre-perfused to remove placental and umbilical cord blood. The perfusion tubing in the two umbilical cord arteries were kept and used for perfusion decellularization. Decellularization is achieved by use of a decellularization solution, said solution comprising phosphate-buffered saline (PBS) and 1% Triton X-100, 0.5% SDS, which is sequentially infused into the placenta via the arteries of the umbilical cord. Residual detergent following decellularization is rinsed off using a PBS solution. Progress of decellularization is monitored by visual inspection for morphology changes of the placenta. Perfusion decellularization is set up using a peristaltic pump with controlled flow rate between 8 to 16 ml/min, with a second, linked peristaltic pump to drain the flow-through of solution into a waste bin. Each step of perfusion utilizes approximately 10 to 20 L of medium over the course of between 8 and 24 hrs. After completing the last PBS perfusion, the decellularized placental vascular scaffold is preserved in PBS with antibiotics (1% penicillin+streptomycin) at 4.degree. C. in, e.g., a stainless pan or desiccator (VWR). Two grams of said decellularized tissue is placed in a roller tissue culture tube in a volume of 20 ml RPMI media with 10% FCS together with antibiotic/antimycotic. 2 million PC-3 cells are added to the tissue culture together with 2 million Wharton's Jelly derived mesenchymal stem cells. Said matrix is cultured for a period of 24 hours, subsequent to which HUVEC cells are seeded at a concentration of 2 million cells per culture. After 48 hours of culture the composite tissue is irradiated at 12 Gy and utilized for vaccination of patients alone, or together with adjuvant.

In various other specific embodiments, the composite cellular vaccine comprise one or more cell types, wherein said one or more cell types are, or comprise, differentiated cells, e.g., one or more of endothelial cells, epithelial cells, dermal cells, endodermal cells, mesodermal cells, fibroblasts, osteocytes, chondrocytes, natural killer cells, dendritic cells, hepatic cells, pancreatic cells, or stromal cells. In various more specific embodiments, said differentiated cells are, in a preferred embodiment transformed cells comprising salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue- sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells, cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

In one embodiment, the oncogenic function desired to replicate the tumor is production of a protein or polypeptide, in specific embodiments, said protein or polypeptide is a cytokine or a peptide comprising an active part thereof. In more specific embodiments, said cytokine is adrenomedullin (AM), angiopoietin (Ang), bone morphogenetic protein (BMP), brain-derived neurotrophic factor (BDNF), epidermal growth factor (EGF), erythropoietin (Epa), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (PIGF), platelet-derived growth factor (PDGF), thrombopoietin (Tpo), transforming growth factor alpha (TGF-α), TGF-β, tumor necrosis factor alpha (TNF-α), vascular endothelial growth factor (VEGF), or a Wnt protein. In a more specific embodiment of said organoids, an individual said organoid, e.g., an organoid comprising 1×10⁸ cells, produces at least 1.0 to 10 μLIM said cytokine in in vitro culture in growth medium over 24 hours. In other specific embodiments, said protein or polypeptide is a soluble receptor for AM, Ang, BMP, BDNF, EGF, Epa, FGF, GNDF, G-CSF, GM-CSF, GDF-9, HGF, HDGF, IGF, migration-stimulating factor, GDF-8, MGF, NGF, PIGF, PDGF, Tpo, TGF-.alpha., TGF-.beta., TNF-.alpha., VEGF, or a Wnt protein. In a more specific embodiment of said organoids, an individual organoid, e.g., an organoid comprising 1×10* cells, produces at least 1.0 to 10 μLIM of said soluble receptor in in vitro culture in growth medium over 24 hours. In other specific embodiments, said protein or polypeptide is an interleukin, e.g., interleukin-1 alpha (IL-1α), IL-Iβ, IL-IF1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, both IL-12 alpha and beta subunits, IL-13, IL-14, IL-15, IL-16, IL-17A, IL-17B, IL-17C, IL-170, IL-17E, IL-17F isoform 1, IL-17F isoform 2, IL-18, IL-19, IL-20, IL-21, IL-22, IL-23 p19 subunit, IL-23 p40 subunit, IL-23 p19 subunit and IL-23 p40 subunit together, IL-24, IL-25, IL-26, IL-27B, IL-27-p28, IL-27B and IL-27-p28 together, IL-28A, IL-28B, IL-29, IL-30, IL-31, IL-32, IL-33, IL-34, IL-35, IL-36α, IL-36β, IL-36γ. In a more specific embodiment of said composite vaccine, I.times.10.sup.8 cells, produces at least 1.0 to 10 μM of said interleukin in in vitro culture in growth medium over 24 hours.

In a further preferred embodiment, the composite cellular vaccine is administered as an immunogenic preparation further comprises a pharmaceutical excipient and/or an immune modulator. Any known inert pharmaceutically acceptable carrier and/or excipient may be added to the composition. Formulation of medicaments, and the use of pharmaceutically acceptable excipients are known and customary in the art and for instance described in Remington; The Science and Practice of Pharmacy, 22^{nd} Edition 2005, University of Sciences in Philadelphia.

Any known immune modulator, may be added to the composition. Preferably, the immune modulator is an adjuvant stimulating type 1 immunity. In one embodiment the adjuvant is an oil-in-water emulsion such as incomplete Freunds Adjuvants, MONTANIDE.™. ISA51(Seppic, France), MONTANIDE™ 720 (Seppic, France). This type of medicament may be administered as a single administration. Alternatively, the administration of the composite vaccine as earlier herein defined and/or an adjuvant may be repeated if needed and/or distinct peptides and/or distinct adjuvants may be sequentially administered.

Particularly preferred adjuvants are those that are known to act via the Toll-like receptors. Adjuvants that are capable of activation of the innate immune system, can be activated particularly well via Toll like receptors (TLR's), including TLR's 1-10 and/or via a RIG-1(Retinoic acid-inducible gene-1) protein and/or via an endothelin receptor. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heatshock proteins from bacteria or from the host, and Mycobacteriallipoarabinomannans. TLR3 may be activated by dsRNA, in particular of viral origin, or by the chemical compound poly(I:C). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLRS may be activated with bacterial flagellae or flagellin. TLR6 may be activated by mycobacterial lipoproteins and group B Streptococcus heat labile soluble factor (GBS-F) or Staphylococcus modulins. TLR7 may be activated by imidazoquinolines and derivatives. TLR9 may be activated by unmethylated CpG DNA or chromatin—IgG complexes. In particular TLR3, TLR4, TLR7 and TLR9 play an important role in mediating an innate immune response against viral infections, and compounds capable of activating these receptors are particularly preferred for use in the invention. Particularly preferred adjuvants comprise, but are not limited to, synthetically produced compounds comprising dsRNA, poly(I:C), unmethylated CpG DNA which trigger TLR3 and TLR9 receptors, IC31, a TLR9 agonist, IMSAVAC, a TLR4 agonist.

In another preferred embodiment, the adjuvants are physically linked to a peptide as earlied defined herein. Physical linkage of adjuvants and costimulatory compounds or functional groups, to the HLA class I and HLA class II epitope comprising peptides provides an enhanced immune response by simultaneous stimulation of antigen presenting cells, in particular dendritic cells, that internalize, metabolize and display antigen. Another preferred immune modifying compound is a T cell adhesion inhibitor, more preferably an inhibitor of an endothelin receptor such as BQ-788 (Buckanovich R Jet al, Ishikawa K, PNAS (1994) 91:4892). BQ-788 is N-cis-2,6-dimethylpiperidinocarbonyi-L-gamma-methylleucyi-D -1-methoxycarbonyltryptophanyi-D-norleucine. However any derivative of BQ-788 or modified BQ-788 compound is also encompassed within the scope of this invention.

Furthermore, the use of APC (co)stimulatory molecules, as set out in W099/61065 and in W003/084999, in combination with a peptide present in the medicament used in the invention is preferred. In particular the use of 4-1-BB and/or CD40 ligands, agonistic antibodies or functional fragments and derivates thereof, as well as synthetic compounds with similar agonistic activity are preferably administered separately or combined with a peptide present in the medicament to subjects to be treated in order to further stimulate the mounting an optimal immune response in the subject. In a preferred embodiment, the adjuvant comprises an exosome, a dendritic cell, monophosphoryllipid A and/or CpG nucleic acid.

In a preferred embodiment, a medicament comprises a peptide or a composition as earlier defined herein and an adjuvant selected from the group consisting of: oil-in water emulsions (MONTANIDE™ ISA51, MONTANIDE™ ISA 720), an adjuvant known to act via a Toll-like receptor, an APC-costimulatory molecule, an exosome, a dendritic cell, monophosphoryllipid A and a CpG nucleic acid.

Ways of administration are known and customary in the art are for instance described in Remington; The Science and Practice of Pharmacy, 21⁵¹ Edition 2005, University of Sciences in Philadelphia. Composite cellular vaccine compositions and pharmaceutical compositions and medicaments of the invention are preferably formulated to be suitable for intravenous or subcutaneous, or intramuscular administration, although other administration routes can be envisaged, such as mucosal administration or intradermal and/or intracutaneous administration, e.g. by injection. Intradermal administration is preferred herein. Advantages and/or preferred embodiments that are specifically associated with intradermal administration are later on defined in a separate section entitled “intradermal administration”.

It is furthermore encompassed by the present invention that the administration of the composite cellular vaccine and/or at least one composition of the invention may be carried out as a single administration. Alternatively, the administration of at least one peptide and/or at least one composition may be repeated if needed and/or distinct peptides and/or compositions of the invention may be sequentially administered.

Any way of administration of the composite cellular vaccine composition or medicament of the invention may be used. The composition or medicament of the invention may be formulated to be suitable for intravenous or subcutaneous, or intramuscular administration, although other administration routes may be envisaged, such as mucosal or intradermal and/or intracutaneous administrations, e.g. by injection.

In addition, a preferred embodiment comprises delivery of the composite cellular vaccine, with or without additional immune stimulants such as TLR ligands and/or anti CD40/anti-4-1BB antibodies in a slow release vehicle such as mineral oil (e.g. MONTANIDE™ ISA 51) or PLGA. Alternatively, a peptide of the invention may be delivered by intradermally, e.g. by injection, with or without immune stimulants (adjuvants). Preferably for intradermal delivery a peptide of the invention is administered in a composition consisting of the peptides and one or more immunologically inert pharmaceutically acceptable carriers, e.g. buffered aqueous solutions at physiological ionic strength and/or osmolarity (such as e.g. PBS).

1. A cancer vaccine comprised of a tissue composite, comprising one or more types of cells, and comprising decellularized placental vascular scaffold, one or more cancer cell lines, and at least one cell type associated with tumors in vivo.

2. The cancer vaccine of claim 1, wherein said tissue composite contains an antigenic mixture different from said cancer cell lines grown in vitro in two dimensional culture.

3. The cancer vaccine of claim 1, wherein said one or more types of cells comprise natural killer (NK) cells, dendritic cells, thymocytes, lymphoid cells, epithelial reticular cells, thymic stromal cells, follicular cells, cells that express thyroglobulin, thyroid epithelial cells, fibroblasts, monocytes, type 2 monocytes, parafollicular cells, comprise stem cells or progenitor cells.

4. The cancer vaccine of claim 3, wherein said stem cells or progenitor cells are embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDAC.®.), umbilical cord stem cells, amniotic fluid stem cells, amnion derived adherent cells (AMDACs), osteogenic placental adherent cells (OPACs), adipose stem cells, limbal stem cells, dental pulp stem cells, placental stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells, hematopoietic stem cells or hematopoietic progenitor cells, tissue culture plastic-adherent CD34⁻, CD10⁺, CD105⁺, and CD200⁺ placental stem cells, or side population stem cells.

5. The cancer vaccine of claim 3, wherein said embryonic stem cells are totipotent and express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

6. The cancer vaccine of claim 3, wherein said cord blood stem cells are multipotent and capable of differentiating into endothelial, smooth muscle, and neuronal cells.

7. The cancer vaccine of claim 3, wherein said cord blood stem cells are identified based on expression of one or more antigens selected from a group comprising: SSEA-3, SSEA-4, CD9, CD34, c-kit, OCT-4, Nanog, and CXCR-4.

8. The cancer vaccine of claim 3, wherein said cord blood stem cells do not express one or more markers selected from a group comprising of: CD3, CD34, CD45, and CD11b.

9. The cancer vaccine of claim 3, wherein said placental stem cells are isolated from the placental structure.

10. The cancer vaccine of claim 9, wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

11. The cancer vaccine of claim 9, wherein said placental stem cells are mesenchymal stem cells.

12. The cancer vaccine of claim 3, wherein said bone marrow stem cells comprise of bone marrow mononuclear cells.

13. The cancer vaccine of claim 3, wherein said bone marrow stem cells comprise of bone marrow mesenchymal stem cells expressing CD73.

14. The cancer vaccine of claim 3, wherein said bone marrow stem cells are selected based on the ability to differentiate into one or more of the following cell types: endothelial cells, smooth muscle cells, and neuronal cells.

15. The cancer vaccine of claim 3, wherein said bone marrow stem cells comprise of bone marrow mononuclear cells, wherein said bone marrow stem cells are selected based on expression of one or more of the following antigens:
CD34, c-kit, flk-1, Stro-1, CD105, CD73, CD31, CD146, vascular endothelial-cadherin, CD133 and CXCR-4.

16. The cancer vaccine of claim 3, wherein said placental stem cells are mesenchymal in morphology.

17. The cancer vaccine of claim 16, wherein said placental cell expresses one or more cytokines associated with a cancer growth, metastasis, angiogenesis or tissue invasiveness.

18. The cancer vaccine of claim 17, wherein said cytokines associated with a cancer growth, metastasis, angiogenesis or tissue invasiveness are selected from a group comprising of CS-6, IL-6, IL-8, SDF-1, CXCL5, VEGF, CXCL6, COL4A4, MMP13, CYP7B1, ADAMDEC1, SLC6A1, CXCL1, PF4V1, CXCL3, CH25H, SFRP2, MMP1, DARC, HCK, bFGF, ERC2, CLIC6, and BCL8.

19. The cancer vaccine of claim 18, wherein said placental stem cells exhibit expression of about 0.0-2,200.0 pg/ml of each of the one or more cytokines in a 75% confluent culture in a T 175 flask.

20. The cancer vaccine of claim 18, wherein said placental cell exhibits expression of between 200.0-1900.0 pg/ml of IL-6 in a 75% confluent culture in a T 175 flask.

21. The cancer vaccine of claim 18, wherein, said cell expresses between 350.0-2,000.0 pg/ml of bFGF in a 75% confluent culture in a T 175 flask.

22. The cancer vaccine of claim 18, wherein said cell expresses between 500.0-2,500.0 pg/ml of VEGF in a 75% confluent culture in a T 175 flask.

23. The cancer vaccine of claim 18, wherein said cell expresses between 140.0-1,500.0 pg/ml of SDF-1in a 75% confluent culture in a T 175 flask.

24. The cancer vaccine of claim 3, wherein said adipose stem cell expresses markers selected from a group comprising of: CD13, CD29, CD44, CD63, CD73, CD90, CD166, Aldehyde dehydrogenase (ALDH), and ABCG2.

25. The cancer vaccine of claim 24, wherein said adipose tissue derived stem cells are a population of purified mononuclear cells extracted from adipose tissue capable of proliferating in culture for more than 1 month.

26. The cancer vaccine of claim 1, wherein said wherein said cells are differentiated cells either transformed oncologically or not transformed.

27. The cancer vaccine of claim 26, wherein said differentiated cells comprise endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, reticulocytes, megakaryocytes, monocytes, connective tissue macrophages. epidermal Langerhans cells, dendritic cells, microglial cells, neutrophils, eosinophils, basophils, mast cell, helper T cells, suppressor T cells, cytotoxic T cell, natural Killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

28. The cancer vaccine of claim 27, wherein said cell population is oncologically transformed by means selected from a group comprising:
a) transfection with an oncogene;
b) transfection telomerase; and
c) transfection with a combination of an oncogene and telomerase.

29. The cancer vaccine of claim 28, wherein said cell population is immortalized by means of transfection with an oncogene selected from a group of oncogenes comprising:
a) abI;
b) Af4/hrx;
c) akt-2;
d) alk;
e) alk/npm;
f) amII;
g) amII/mtg8;
h) bcI-2, 3, 6;
i) bcr/abI;
j) c-myc;
k) dbI;
l) dek/can;
m) E2A/pbxI;
n) egfr;
o) enl/hrx;
p) erg/TLS;
q) erbB;
r) erbB-2;
s) ets-1;
t) ews/fli-1;
u) fms;
v) fos;
w) fps;
x) gli;
y) gsp;
z) gsp;
aa) HER2/new;
ab)hoxII;
ac) hst;
ad) IL-3;
ae) int-2;
af) jun;
ag) kit;
ah) KS3;
ai) K-sam;
aj) Lbc;
ak) Ick;
al) Imol,Imo-2;
am) L-myc;
an) IyI-1;
ao) Iyt-10;
ap)Iyt-10/C alpha 1;
aq) mas; ar) mdm-2;
as) mil;
at) mas;
au) mtg8/amII;av) myb;
aw) MYHII;
ax) new;
ay) N-myc;
az) ost;
ba) pax-5;
bb) pbxI/E2a;
bc) pim-1;
bd) PRAD-1;
be) rat
bf) RAR/PML;
bg) RasH, K, N;
bh) rel/nrg;
bi) ret;
bj) rhoml, rhom2;
bk) ros;
bl) ski;
bm) sis;
bn) set/can;
bo) src;
bp) Tall,tal2;
bq) tan-1;
br) TiamI;
bs) TSC2; and
bt) trk.

30. The cancer vaccine of claim 1, wherein said cells have been genetically engineered to produce a protein or polypeptide not naturally produced by the cell, or have been genetically engineered to produce a protein or polypeptide in an amount greater than that naturally produced by the cell, wherein said cellular composition comprises differentiated cells.

31. The cancer vaccine of claim 30, wherein said protein or polypeptide is a cytokine or a peptide comprising an active part thereof.

32. The cancer vaccine of claim 31, wherein said cytokine is adrenomedullin (AM), angiopoietin (Ang), bone morphogenetic protein (BMP), brain-derived neurotrophic factor (BDNF), epidermal growth factor (EGF), erythropoietin (Epa), fibroblast growth factor (FGF), glial cell line-derived neurotrophic factor (GNDF), granulocyte colony stimulating factor (G-CSF), granulocyte-macrophage colony stimulating factor (GM-CSF), growth differentiation factor (GDF-9), hepatocyte growth factor (HGF), hepatoma derived growth factor (HDGF), insulin-like growth factor (IGF), migration-stimulating factor, myostatin (GDF-8), myelomonocytic growth factor (MGF), nerve growth factor (NGF), placental growth factor (PIGF), platelet-derived growth factor (PDGF), thrombopoietin (Tpo), transforming growth factor alpha (TGF-a), TGF-, tumor necrosis factor alpha (TNF-a), vascular endothelial growth factor (VEGF), or a Wnt protein.

33. The cancer vaccine of claim 8, wherein said protein or polypeptide is selected from a group comprising of AM, Ang, BMP, BDNF, EGF, Epa, FGF, GNDF, G-CSF, GM-CSF, GDF-9, HGF, HDGF, IGF, migration-stimulating factor, GDF-8, MGF, NGF, PIGF, PDGF, Tpo, TGF-a, TGF-, TNF-a, VEGF, or a Wnt protein; an interleukin; a soluble receptor for IL-1α, IL-1β, IL-1F1, IL-1F2, IL-1F3, IL-1F4, IL-1F5, IL-1F6, IL-1F7, IL-1F8, IL-1F9, IL-2, IL-3, IL-4, IL-5, IL-6, IL-7, IL-8, IL-9, IL-10, IL-11, IL-12 35 kDa alpha subunit, IL-12 40 kDa beta subunit, IL-13, IL-14, IL-15, IL-16, IL-17A, IL-17B, IL-17C, IL-170, IL-17E, IL-17F isoform 1, IL-17F isoform 2, IL-18, IL-19, IL-20, IL-21, IL-22, IL-23 p19 subunit, IL-23 p40 subunit, IL-24, IL-25, IL-26, IL-27B, IL-27-p28, IL-28A, IL-28B, IL-29, IL-30, IL-31, IL-32, IL-33, IL-34, IL-35, IL-36α, IL 36β, IL-36γ; an interferon (IFN); a soluble receptor for IFN-α, IFN-β, IFN-γ, IFN-Aλ1, IFN-λ2, IFN-λ3, IFN-K, IFN-ε, IFN-κ, IFN-_{T}, IFN-δ, or IFN-ζ, IFN-ω, or IFN-v; insulin or proinsulin; a receptor for insulin; leptin (LEP).

34. An in vivo tumor immunogenic composite comprising tumor cells seeded on a matrix combined with antigen presenting cells.

35. A method of producing antigen presenting cell derived exosomes loaded with tumor peptides comprising the steps of:
a) selecting a matrix;
b) seeding said matrix with tumor cells;
c) seeding said matrix with antigen presenting cells in a manner allowing for uptake of tumor antigens from said seeded tumor cells onto said seeded antigen presenting cells; and
d) collecting said exosomes.
